(19) United States
(12) Patent Application Publication
(10) Pub. No.: US 2013/0184530 A1
(43) Pub. Date: Jul. 18, 2013

(54) ENDOSCOPE APPARATUS AND BRIGHTNESS CONTROL METHOD

(71) Applicant: Olympus Corporation, Tokyo (JP)

(72) Inventor: Seigo ON, Tokyo (JP)

(73) Assignee: OLYMPUS CORPORATION, Tokyo (JP)

(21) Appl. No.: 13/738,178

(22) Filed: Jan. 10, 2013

(30) Foreign Application Priority Data
Jan. 13, 2012 (JP) 2012-005313

(51) Int. Cl. A61B 1/00; A61B 1/06

(52) U.S. Cl. CPC A61B 1/00188; A61B 1/06
USPC 600/168

(57) ABSTRACT

An endoscope apparatus includes an objective optical system that is configured so that an observation magnification in at least a center part of an observation range is variable, a magnification control section that controls the observation magnification, and a brightness control section that performs a photometric process that attaches weight to a center part of an image that corresponds to the center part of the observation range when the observation magnification is a second magnification that is higher than a first magnification as compared with a case where the observation magnification is the first magnification, and adjusts the brightness of the image based on the result of the photometric process.

Japanese Patent Application No. 2012-005313 filed on Jan. 13, 2012, is hereby incorporated by reference in its entirety.

### BACKGROUND

The present invention relates to an endoscope apparatus, a brightness control method, and the like.

A wide-angle optical system has been used for an endoscope apparatus in order to observe the object over a wide range. For example, JP-A-2010-117665 discloses a wide-angle optical system that is configured so that the observation state can be switched using a variable aperture between a state in which the center part of the observation range and the part of the observation range positioned outside the center part can be simultaneously observed, and a state in which only the center part of the observation range can be observed. The state in which the center part of the observation range and the part of the observation range positioned outside the center part can be simultaneously observed is effective for observing the back side of the folds of a large intestine using an endoscope apparatus, and may make it possible to find a lesion that is otherwise missed.

### SUMMARY

According to one aspect of the invention, there is provided an endoscope apparatus comprising:

an objective optical system that is configured so that an observation magnification in at least a center part of an observation range is variable;

a magnification control section that controls the observation magnification; and

a brightness control section that performs a photometric process that attaches weight to a center part of an image that corresponds to the center part of the observation range when the observation magnification is a second magnification that is higher than a first magnification as compared with a case where the observation magnification is the first magnification, and adjusts brightness of the image based on a result of the photometric process.

According to another aspect of the invention, there is provided a brightness control method comprising:

controlling an observation magnification of an objective optical system that is configured so that the observation magnification in at least a center part of an observation range is variable; and

performing a photometric process that attaches weight to a center part of an image that corresponds to the center part of the observation range when the observation magnification is a second magnification that is higher than a first magnification as compared with a case where the observation magnification is the first magnification, and adjusting brightness of the image based on a result of the photometric process.

### BRIEF DESCRIPTION I/F THE DRAWINGS

FIG. 1 illustrates a configuration example of an endoscope apparatus according to a first embodiment.

FIG. 2 illustrates a configuration example of a captured image.

FIG. 3 illustrates a detailed configuration example of an objective optical system.

FIG. 4 illustrates a detailed configuration example of a magnification control section according to the first embodiment.

FIG. 5 illustrates a detailed configuration example of a brightness control section.

FIG. 6 is a view illustrating a sampling process.

FIG. 7 is a view illustrating a sampling process.

FIG. 8 is a view illustrating a sampling process.

FIG. 9 illustrates a detailed configuration example of a brightness section.

FIG. 10 illustrates a configuration example of an endoscope apparatus according to a second embodiment.

FIG. 11 illustrates a detailed configuration example of a magnification control section according to the second embodiment.

### DESCRIPTION I/F EXEMPLARY EMBODIMENTS

According to one embodiment of the invention, there is provided an endoscope apparatus including:

an objective optical system that is configured so that an observation magnification in at least a center part of an observation range is variable;

a magnification control section that controls the observation magnification; and

a brightness control section that performs a photometric process that attaches weight to a center part of an image that corresponds to the center part of the observation range when the observation magnification is a second magnification that is higher than a first magnification as compared with a case where the observation magnification is the first magnification, and adjusts brightness of the image based on a result of the photometric process.

According to the endoscope apparatus, the photometric process that attaches weight to the center part of the image is performed when the observation magnification is the second magnification that is higher than the first magnification as compared with the case where the observation magnification is the first magnification. This makes it possible to implement a brightness control process that is optimum for the observation target.

Exemplary embodiments of the invention are described below. Note that the following exemplary embodiments do not in any way limit the scope of the invention laid out in the claims. Note also that all of the elements described in connection with the following exemplary embodiments should not necessarily be taken as essential elements of the invention.

### 1. First Embodiment

### 1.1. Endoscope Apparatus

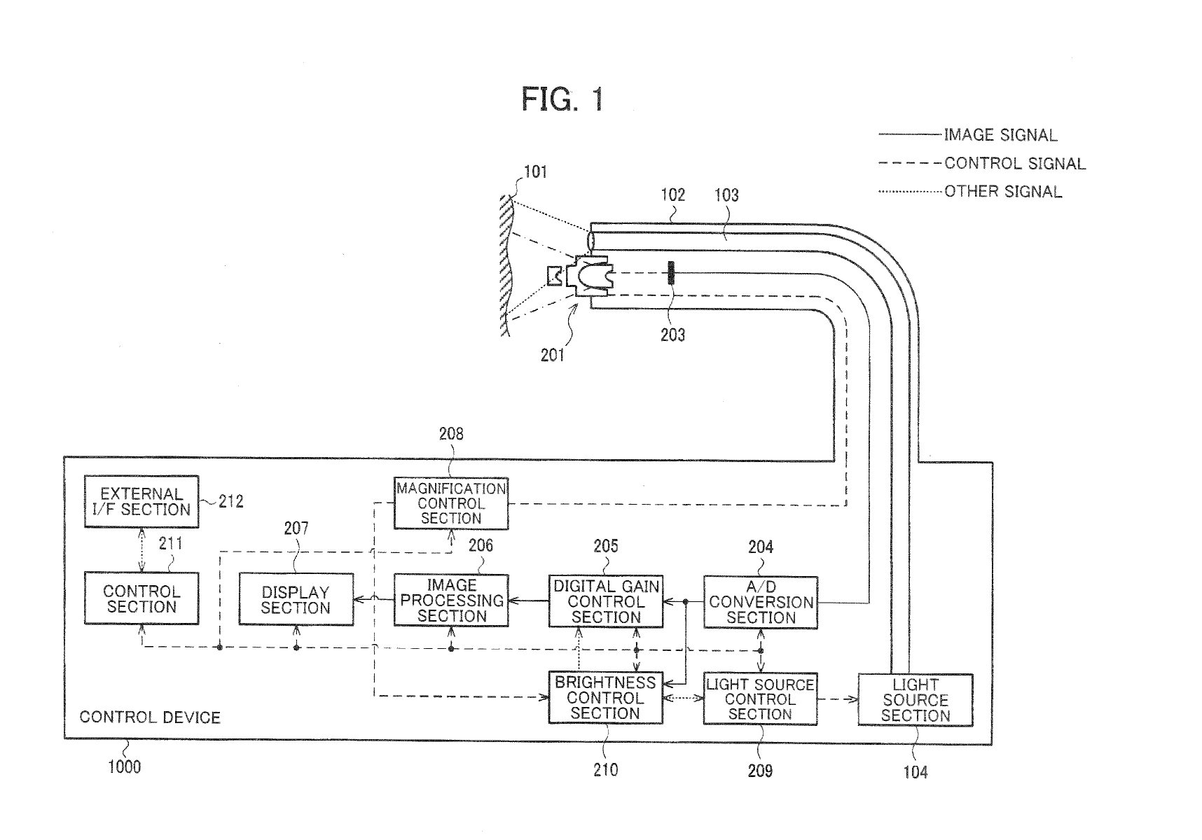

FIG. 1 illustrates a configuration example of an endoscope apparatus according to a first embodiment of the invention. The endoscope apparatus includes an insertion section 102 (imaging section in a broad sense) and a control device 1000 (processor section).

The insertion section 102 captures (images) an object inside a body cavity (e.g., digestive tract). The insertion section 102 includes a light guide 103, an objective optical system 201, and an image sensor 203.

The control device 1000 controls the endoscope apparatus, and performs image processing. The control device 1000 includes a light source section 104, an A/D conversion section 204, a digital gain control section 205, an image processing section 206, a display section 207, a magnification control section 208, a light source control section 209, a brightness control section 210, a control section 211, and an external I/F section 212.

The A/D conversion section 204 is connected to the digital gain control section 205 and the brightness control section 210. The digital gain control section 205 is connected to the display section 207 via the image processing section 206. The magnification control section 208 is connected to the brightness control section 210 and the objective optical system 201. The brightness control section 210 is connected to the digital gain control section 205, and is bidirectionally connected to the light source control section 209. The light source control section 209 is connected to the light source section 104. The control section 211 is bidirectionally connected to the A/D conversion section 204, the digital gain control section 205, the image processing section 206, the display section 207, the magnification control section 208, the light source control section 209, the brightness control section 210, and the external I/F section 212.

The insertion section 102 has an elongated shape and can be curved so that the insertion section 102 can be inserted into a body cavity (body) in order to perform endoscopic examination or treatment. The insertion section 102 can be removed from the control device 1000. The doctor (user or observer in a broad sense) selects the desired scope from a plurality of scopes (insertion sections 102) depending on the objective of examination, attaches the selected scope to the control device 1000, and performs examination or treatment.

Light emitted from the light source section 104 is applied to an object 101 via the light guide 103 that can be curved. The objective optical system 201 is disposed on the end of the insertion section 102, and reflected light from the object 101 enters the image sensor 203 via the objective optical system 201.

The objective optical system 201 is a wide-angle optical system that includes a first optical system for observing a center field of view (i.e., the center part of the observation range), and a second optical system for observing a side field of view (i.e., the part of the observation range other than the center part, or the peripheral part of the observation range). The center field of view and the side field of view can be simultaneously captured using the objective optical system 201. The details of the objective optical system 201 are described later.

Reflected light from the object 101 within the center field of view and reflected light from the object 101 within the side field of view form an optical image on the image sensor 203 via the objective optical system 201. An analog image signal output from the image sensor 203 is transmitted to the A/D conversion section 204. For example, the image sensor 203 is a 3-channel (RGB) image sensor, and outputs 3-channel (RGB) analog image signals. The A/D conversion section 204 converts the analog image signal output from the image sensor 203 into a digital image signal (hereinafter may be referred to as “captured image” or “image”), and transmits the digital image signal to the digital gain control section 205 and the brightness control section 210.

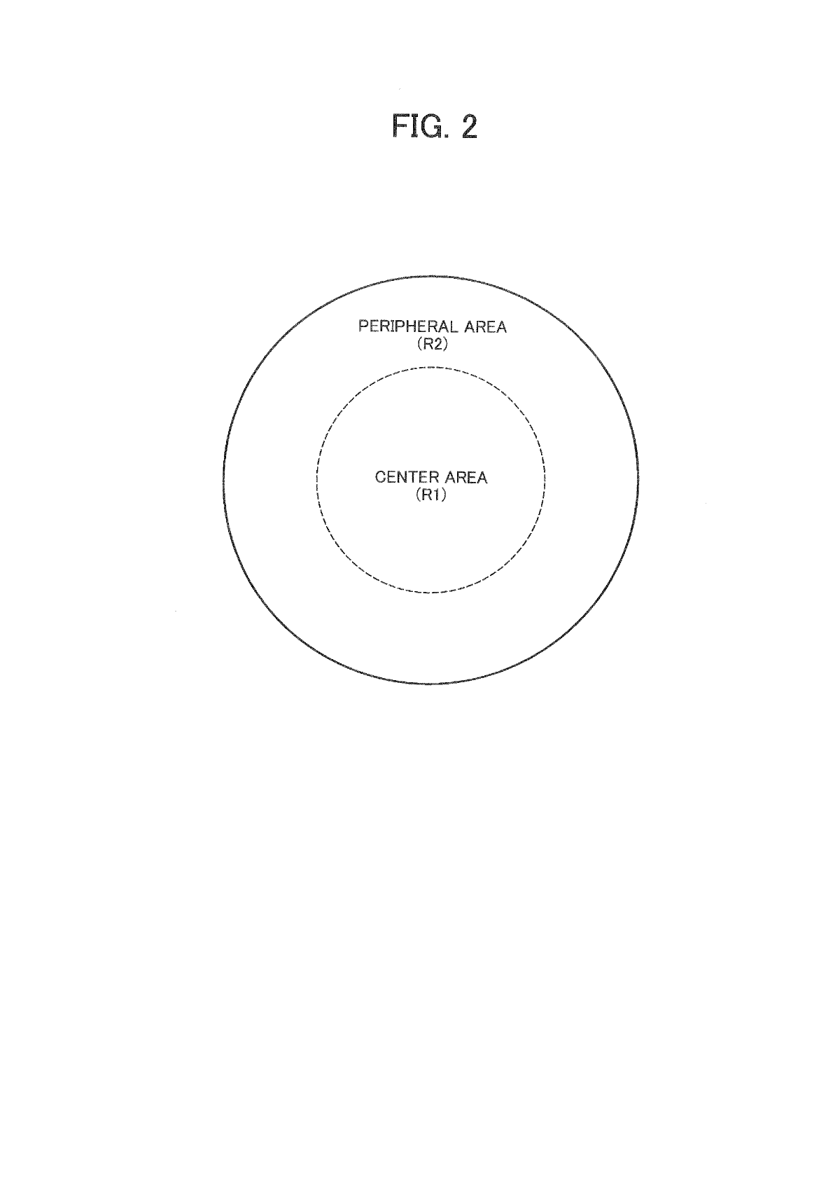

FIG. 2 illustrates a configuration example of an image that is captured according to the first embodiment. A center area R1 (first area in a broad sense) that is the center part of the image corresponds to the center field of view that is the observation range of the first optical system. A peripheral area R2 (second area in a broad sense) that is the part (peripheral part) of the image other than the center part corresponds to the side field of view that is the observation range of the second optical system.

The magnification control section 208 controls the observation magnification (imaging magnification) of the insertion section 102. The details of the magnification control section 208 are described later. The brightness control section 210 performs a brightness control process based on the observation magnification and the captured image. More specifically, the brightness control section 210 performs a brightness control process that attaches weight to the center area R1 of the image when the observation magnification is higher than a given magnification. For example, the brightness control section 210 performs a center-weighted metering process as the brightness control process that attaches weight to the center area R1. Note that the term “center-weighted metering process” used herein refers to a photometric process that is mainly performed on the center part of the observation range, and includes at least a photometric process (i.e., a process that calculates a brightness evaluation value) described herein. The details of the brightness control section 210 are described later. The light source control section 209 adjusts the intensity of light emitted from the light source section 104 under control of the brightness control section 210. The digital gain control section 205 adjusts the brightness (e.g., luminance) of the captured image using a digital gain under control of the brightness control section 210.

The image processing section 206 performs image processing (e.g., known image processing) on the image processed by the digital gain control section 205 under control of the control section 211. For example, the image processing section 206 performs a white balance process, a color management process, a grayscale transformation process, and the like. The image processing section 206 transmits the resulting RGB image to the display section 207. The display section 207 displays the RGB image.

The control section 211 controls each section of the endoscope apparatus. The external I/F section 212 receives operation information input by the doctor, and outputs the operation information to the control section 211.

### 1.2. Objective Optical System

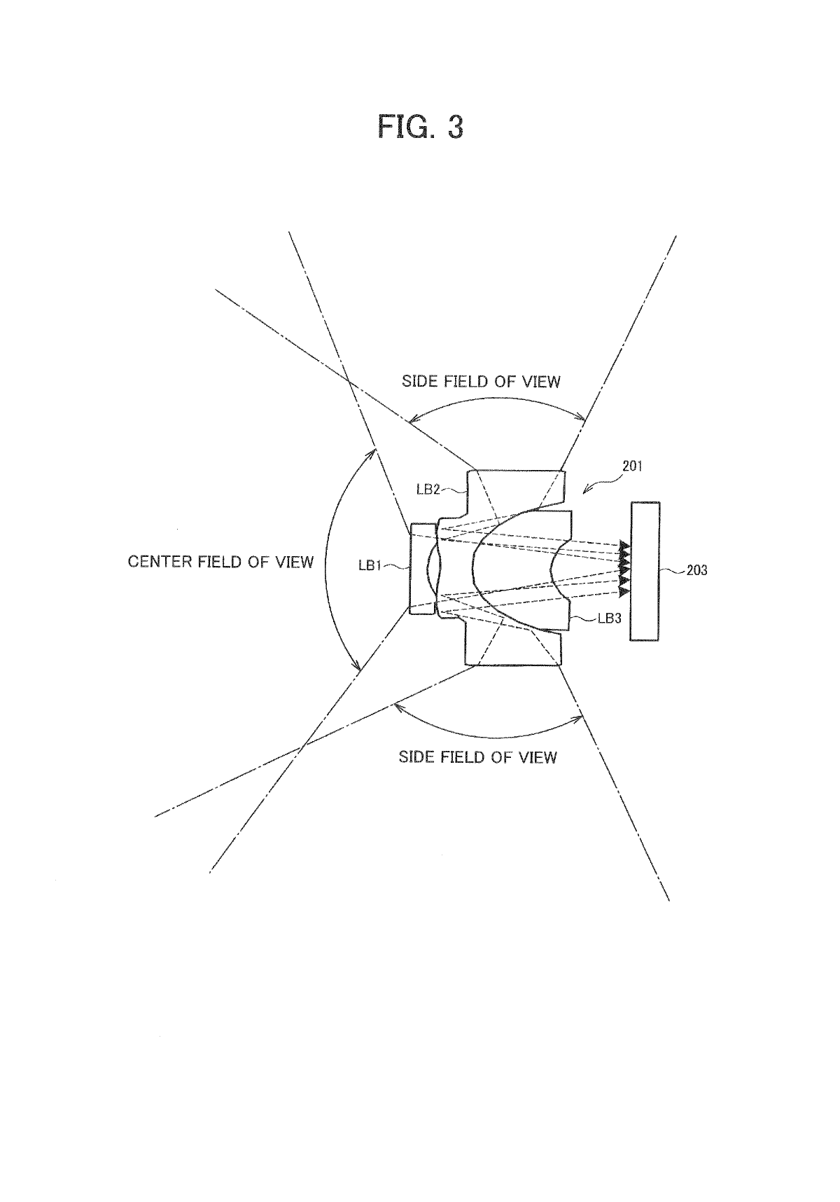

FIG. 3 illustrates a detailed configuration example of the objective optical system 201. The objective optical system 201 includes members LB1 to LB3. Light that has entered the member LB1 from the center field of view that corresponds to the center part of the observation range is refracted by the members LB1 to LB3, and forms an image on the image sensor 203. The optical system that causes light from the center field of view to form an image corresponds to the first optical system. Light that has entered the member LB2 from the side field of view that corresponds to the peripheral part of the observation range is refracted by the members LB2 and LB3, and forms an image on the image sensor 203. The optical system that causes light from the side field of view to form an image corresponds to the second optical system. The magnification control section 208 controls only the observation magnification in the center field of view (first optical system), and does not control the observation magnification in the side field of view (second optical system).

Note that the first optical system and the second optical system need not necessarily be implemented by different elements, but may be implemented by common elements (see FIG. 3). The observation magnification in the side field of view may also be controlled instead of controlling only the observation magnification in the center field of view. In this case, at least the observation magnification in the center field of view may be compared with a given magnification.

### 1.3. Magnification Control Section

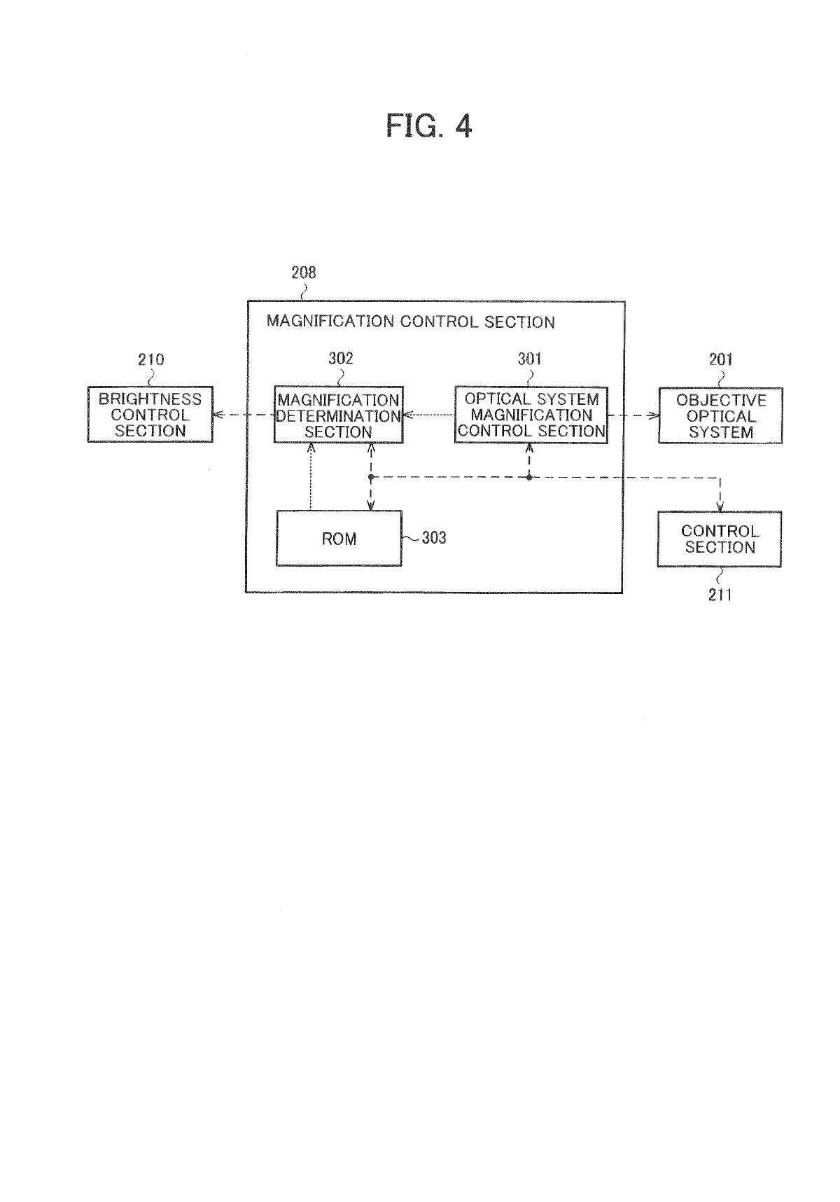

FIG. 4 illustrates a detailed configuration example of the magnification control section 208. The magnification control section 208 includes an optical system magnification control section 301, a magnification determination section 302, and a ROM 303 (memory or storage section in a broad sense).

The optical system magnification control section 301 is connected to the objective optical system 201 and the magnification determination section 302. The magnification determination section 302 is connected to the brightness control section 210. The ROM 303 is connected to the magnification determination section 302. The control section 211 is bidirectionally connected to the optical system magnification control section 301, the magnification determination section 302, and the ROM 303.

The user adjusts the observation magnification via the external I/F section 212. The control section 211 controls the optical system magnification control section 301 based on the information input by the user, and the optical system magnification control section 301 controls the observation magnification of the objective optical system 201. More specifically, the optical system magnification control section 301 adjusts the observation magnification of the first optical system that corresponds to the center field of view.

The magnification determination section 302 acquires the observation magnification and acquires a magnification threshold value (given threshold value) respectively from the optical system magnification control section 301 and the ROM 303 under control of the control section 211. The magnification determination section 302 compares the observation magnification with the magnification threshold value, and transmits the comparison result to the brightness control section 210. Note that the magnification threshold value differs depending on the type of the attached scope (insertion section 102).

### 1.4. Brightness Control Section

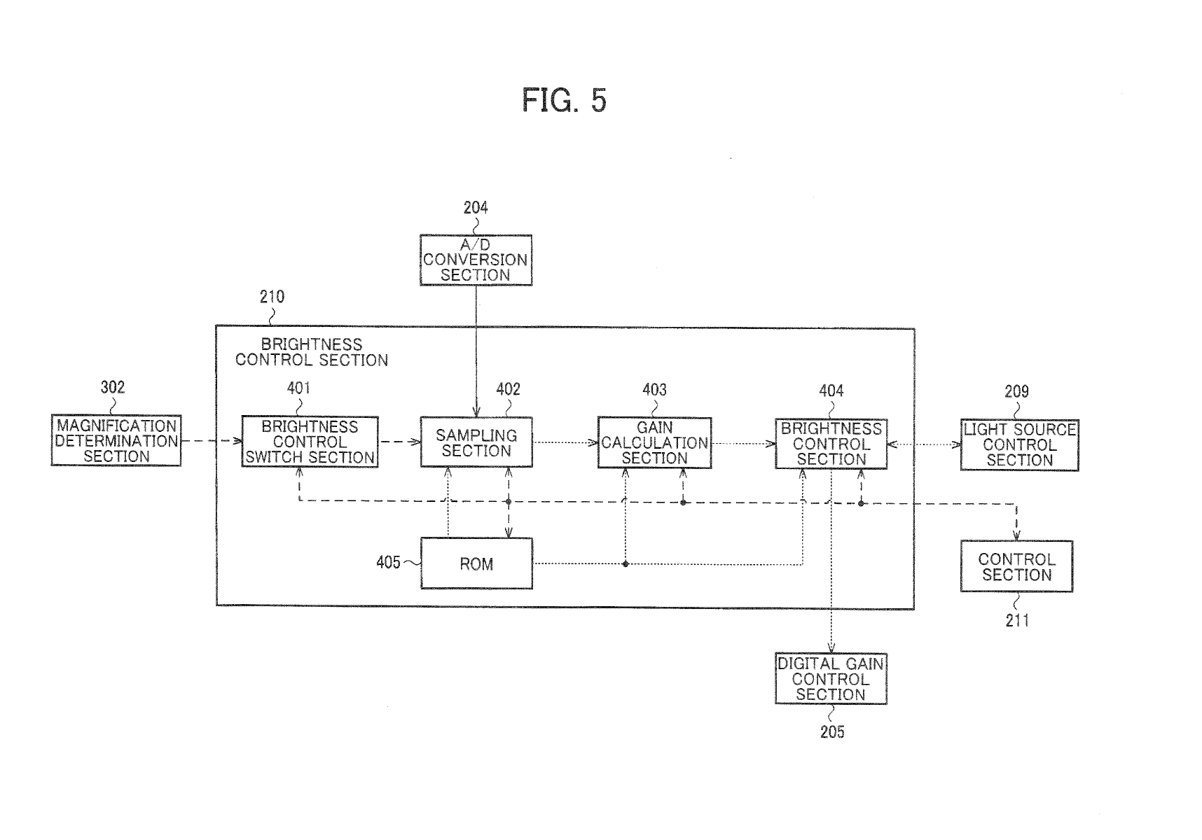

FIG. 5 illustrates a detailed configuration example of the brightness control section 210. The brightness control section 210 includes a brightness control switch section 401, a sampling section 402, a gain calculation section 403, a brightness control section 404, and a ROM 405.

The magnification determination section 302 is connected to the digital gain control section 205 via the brightness control switch section 401, the sampling section 402, the gain calculation section 403, and the brightness control section 404. The brightness control section 404 is bidirectionally connected to the light source control section 209. The ROM 405 is connected to the sampling section 402, the gain calculation section 403, and the brightness control section 404. The A/D conversion section 204 is connected to the sampling section 402. The control section 211 is bidirectionally connected to the brightness control switch section 401, the sampling section 402, the gain calculation section 403, the brightness control section 404, and the ROM 405.

The brightness control switch section 401 switches the brightness control process under control of the control section 211 using information about the comparison between the observation magnification and the magnification threshold value acquired from the magnification determination section 302. More specifically, the brightness control switch section 401 switches the brightness control process to the brightness control process that takes account of the brightness of the entire image (i.e., the center part and the peripheral part of the image) when the observation magnification is smaller than the magnification threshold value. The brightness control switch section 401 switches the brightness control process to the brightness control process that attaches weight to the brightness of the center part of the image when the observation magnification is equal to or larger than the magnification threshold value. The brightness control switch section 401 outputs a signal that instructs to switch the brightness control process to the sampling section 402.

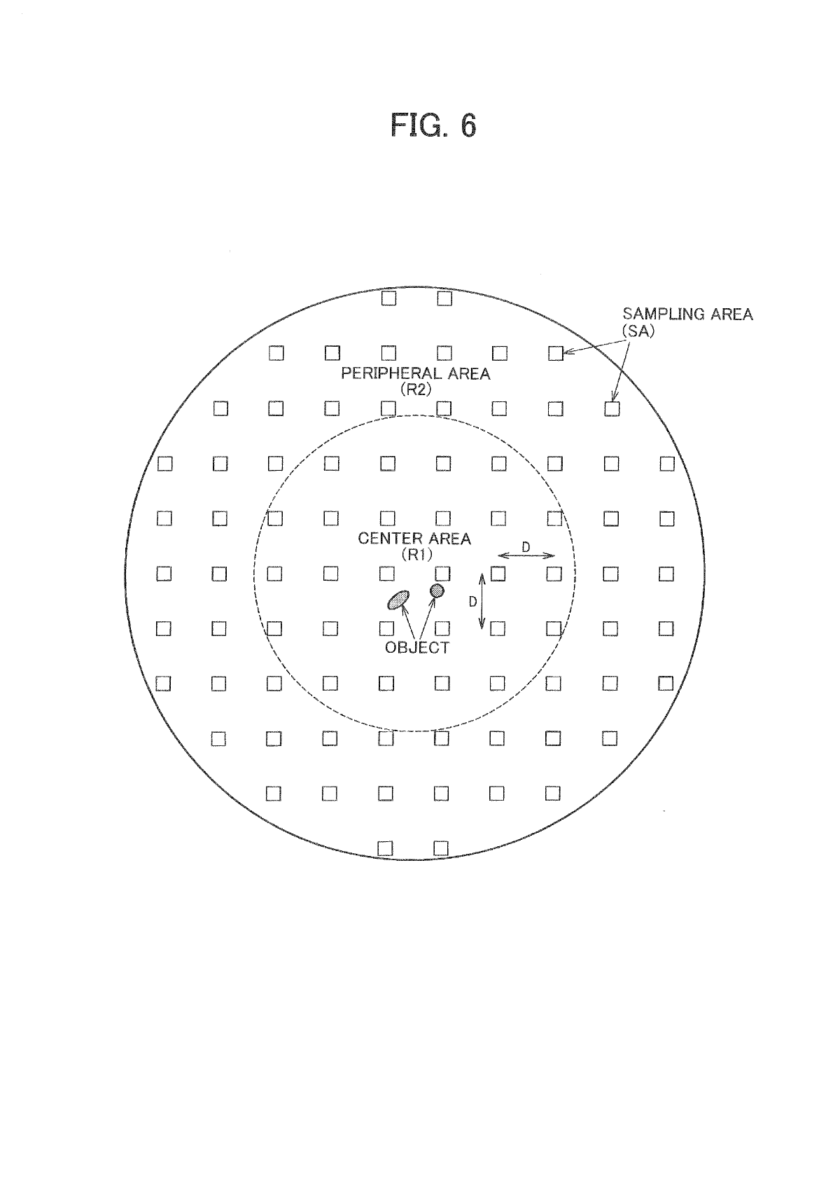

As illustrated in FIG. 6, the sampling section 402 samples brightness information equally from the entire image when the observation magnification is smaller than the magnification threshold value. The following description illustrates an example in which the sampling target is the G signal of the image, and the brightness information is the pixel value. Note that the configuration is not limited thereto.

The sampling section 402 sets sampling target areas SA (hereinafter referred to as “sampling areas”) to the entire image at given intervals D. Note that one sampling area SA may correspond to one pixel, or may be an area that includes a plurality of pixels. The information about the interval D is stored in the ROM 405, and the sampling section 402 reads the information about the interval D from the ROM 405. The sampling section 402 acquires the pixel value from each sampling area SA, and calculates the average value Avg of the acquired pixel values.

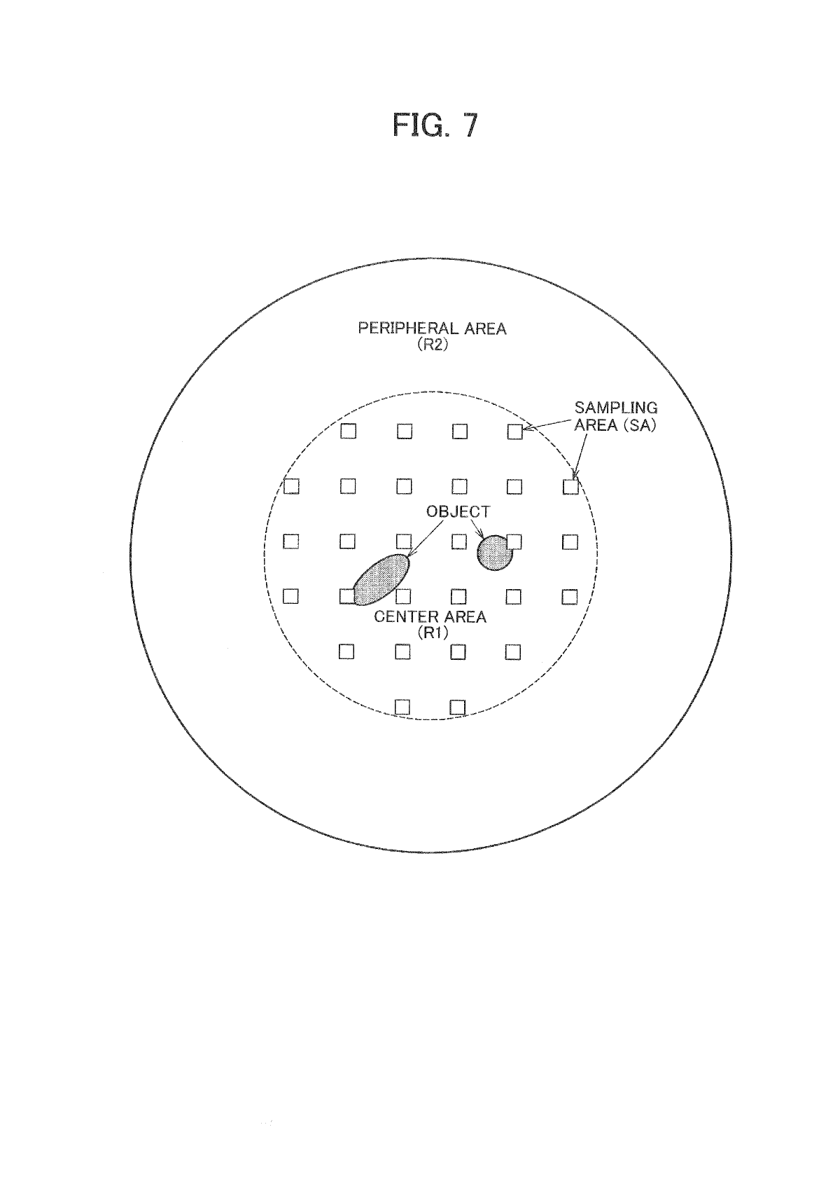

As illustrated in FIG. 7, the sampling section 402 samples the pixel values from only the center area R1 of the image when the observation magnification is equal to or larger than the magnification threshold value. More specifically, the sampling section 402 sets the sampling areas SA to the center area R1 at the given intervals D, acquires the pixel value from each sampling area SA, and calculates the average value Avg of the acquired pixel values. The information about the center area R1 that corresponds to the center part of the image is stored in the ROM 405, and the sampling section 402 reads the information about the center area R1 of the image from the ROM 405. The sampling section 402 transmits the average value Avg to the gain calculation section 403.

Note that the average value (Avg) calculation method when the observation magnification is smaller than the magnification threshold value is not limited to the above method. The average value Avg may be calculated by applying various methods that attach weight to the center part of the image. For example, the average value Avg may be calculated by the following methods (modifications).

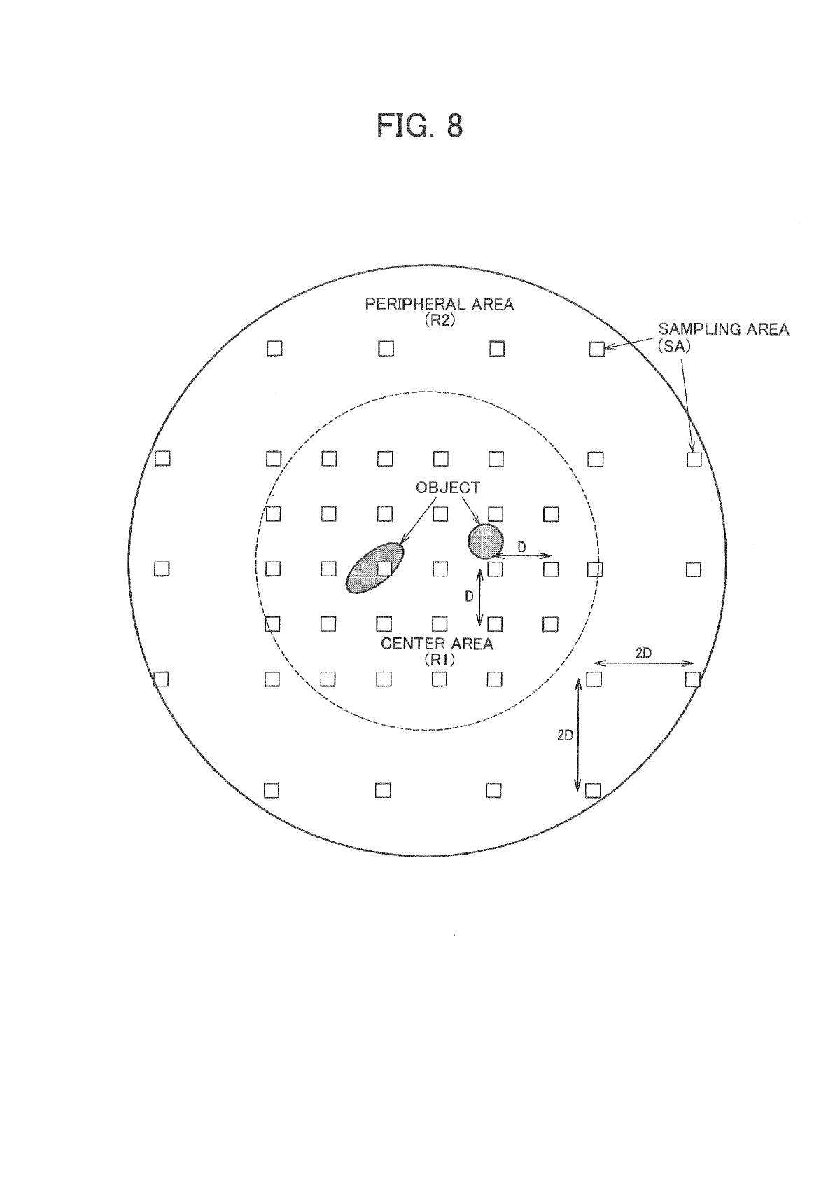

As illustrated in FIG. 8, the sampling section 402 may set the sampling areas SA to the center area R1 and the peripheral area R2 at different intervals (first modification). More specifically, the sampling section 402 may set the sampling areas SA to the center area R1 at the intervals D, and may set the sampling areas SA to the peripheral area R2 at intervals 2D.

Alternatively, the sampling section 402 may multiply the pixel values sampled from the center area R1 and the pixel values sampled from the peripheral area R2 by a different weighting coefficient, and calculate the average value of the resulting pixel values (see the following expression (1)) (second modification). In the expression (1), We is the weighting coefficient for the center area R1, and Ws is the weighting coefficient for the peripheral area R2 (Wc>Ws).

$$Avg = \frac{Wc * {\sum }_{i , j} Vc ( i , j ) + Ws * {\sum }_{k , j} Vs ( k , l )}{m + n} \qquad ( 1 )$$

where, i and j are the horizontal coordinate value and the vertical coordinate value of the sampling target pixel included in the center area R1, respectively, k and l are the horizontal coordinate value and the vertical coordinate value of the sampling target pixel included in the peripheral area R2, respectively, Vc is the pixel value sampled from the center area R1, Vs is the pixel value sampled from the peripheral area R2, m is the number of sampling target pixels included in the center area R1, and n is the number of sampling target pixels included in the peripheral area R2.

Alternatively, the sampling section 402 may set a weighting coefficient that is proportional to the distance from the center pixel of the image (third modification). In this case, the sampling section 402 may decrease the weighting coefficient as the distance from the center pixel of the image to the sampling area SA increases.

The gain calculation section 403 calculates a brightness gain value Gv (see the following expression (2)) using the average value Avg acquired from the sampling section 402 and a brightness control reference value Bv acquired from the ROM 405 under control of the control section 211, and transmits the brightness gain value Gv to the brightness control section 404.

Gv=Bv/Avg (2)

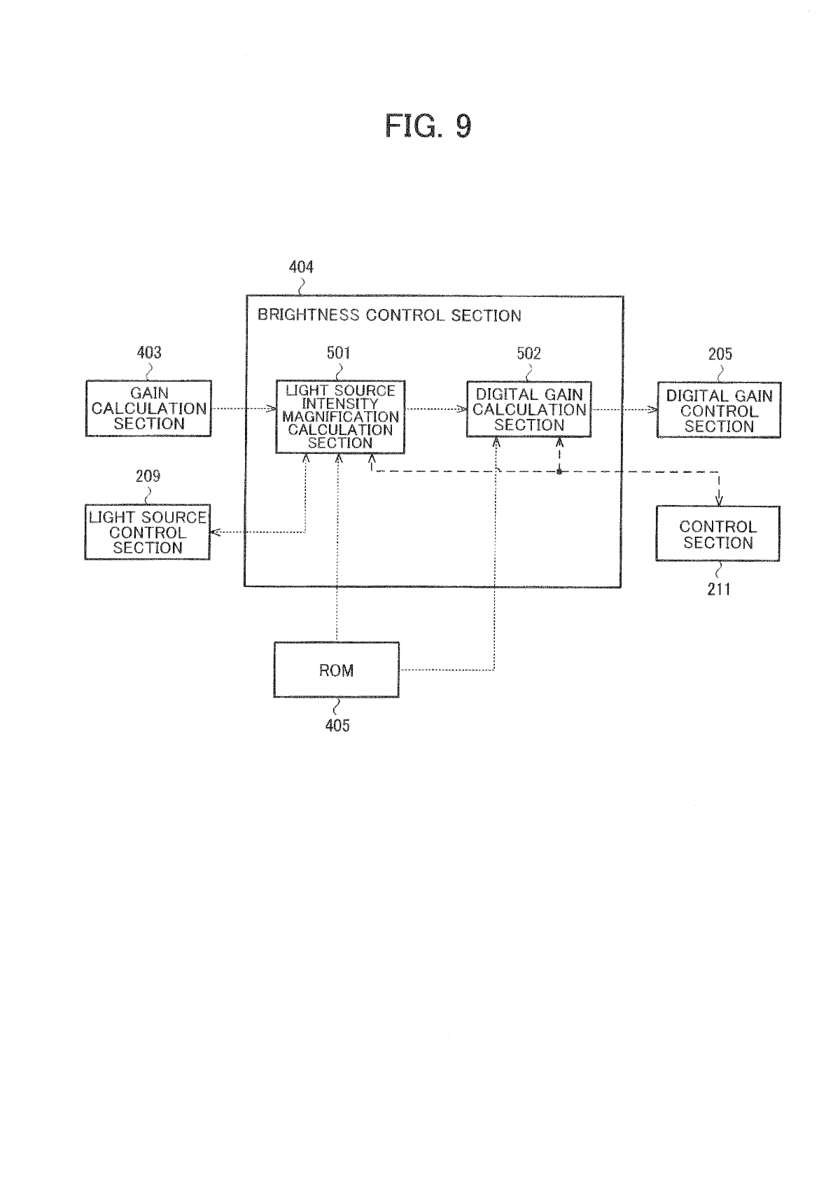

The brightness control section 404 adjusts at least one of the intensity of illumination light and the digital gain of the image based on the brightness gain value Gv to adjust the brightness of the image. FIG. 9 illustrates a detailed configuration example of the brightness control section 404. The brightness control section 404 includes a light source intensity magnification calculation section 501 and a digital gain calculation section 502.

The gain calculation section 403 is connected to the digital gain control section 205 via the light source intensity magnification calculation section 501 and the digital gain calculation section 502. The ROM 405 is connected to the light source intensity magnification calculation section 501 and the digital gain calculation section 502. The light source intensity magnification calculation section 501 is bidirectionally connected to the light source control section 209. The control section 211 is bidirectionally connected to the light source intensity magnification calculation section 501 and the digital gain calculation section 502.

The light source intensity magnification calculation section 501 calculates the maximum magnification Rm (see the following expression (3)) from the current light source intensity Lv and the maximum intensity MAXv of the light source section 104 under control of the control section 211. The current light source intensity Lv is output from the light source control section 209. The maximum intensity MAXv is stored in the ROM 405.

Rm=MAXv/Lv (3)

The light source intensity magnification calculation section 501 compares the maximum magnification Rm with the brightness gain value Gv. The light source intensity magnification calculation section 501 sets the light source intensity magnification to the brightness gain value Gv when the maximum magnification Rm is equal to or larger than the brightness gain value Gv. In this case, the digital gain calculation section 502 sets a digital gain value Dv to “1” under control of the control section 211. The light source intensity magnification calculation section 501 sets the light source intensity magnification to the maximum magnification Rm when the maximum magnification Rm is smaller than the brightness gain value Gv. In this case, the digital gain calculation section 502 calculates the digital gain value Dv using the following expression (4) under control of the control section 211. The light source intensity magnification calculation section 501 transmits the light source intensity magnification to the light source control section 209, and transmits the digital gain value Dv to the digital gain control section 205.

Dv=Gv/Rm (4)

The light source control section 209 adjusts the intensity of light emitted from the light source section 104 based on the light source intensity magnification.

The digital gain control section 205 multiplies the image by the digital gain value Dv (see the following expression (5)) under control of the control section 211. The digital gain control section 205 transmits the resulting image to the image processing section 206.

R′(q,p)=R(q,p)*Dv,

G′(q,p)=G(q,p)*Dv,

B′(q,p)=B(q,p)*Dv (5)

where, q is the horizontal coordinate value of the pixel of the image, p is the vertical coordinate value of the pixel of the image, R, G, and B are the pixel values before multiplication, and R′, G′, and B′ are the pixel values after multiplication.

Although an example in which the brightness control method is automatically switched corresponding to the observation magnification has been described above, the configuration is not limited thereto. For example, the doctor may manually control the brightness control method via the external I/F section 212 corresponding to the observation state.

According to the first embodiment, the endoscope apparatus includes the objective optical system 201, the magnification control section 208, and the brightness control section 210 (see FIG. 1). The objective optical system 201 is configured so that the observation magnification in at least the center part of the observation range is variable. The magnification control section 221 controls the observation magnification. The brightness control section 210 performs the photometric process that attaches weight to the center part of the image that corresponds to the center part of the observation range when the observation magnification is a second magnification that is higher than a first magnification as compared with the case where the observation magnification is the first magnification, and adjusts the brightness of the image based on the result of the photometric process.

In the first embodiment, the observation magnification in the center field of view that corresponds to the center part of the observation range is variable. It is also possible to employ a configuration in which the observation magnification over the entire observation range is variable (as described later in connection with a second embodiment). The first magnification is lower than a given magnification (magnification threshold value), and the second magnification is higher than the given magnification. Note that the configuration is not limited thereto. For example, the brightness control process may be performed so that the degree by which weight is attached to the center part of the observation range continuously changes corresponding to the observation magnification, and is set to be higher when the observation magnification is the second magnification as compared with the case where the observation magnification is the first magnification.

The photometric process that attaches weight to the center part of the image refers to a photometric process in which the brightness of the center part of the image affects the result (e.g., average value Avg) of the photometric process to a large extent as compared with the peripheral part of the image. For example, the photometric process that attaches weight to the center part of the image is a photometric process that attaches weight to the center part of the observation range (center-weighted metering process), or a photometric process that increases the weighting of photometry in the center part of the observation range.

According to the above configuration, since the brightness control process can be adaptively performed corresponding to the observation state of the doctor, it is possible to contribute to an improvement in visibility of a lesion area. More specifically, when the observation magnification is high, it is considered that the doctor desires to closely observe a lesion area (attention area in a broad sense), and observes the lesion area within the center part of the field of view. In this case, the bright center part of the image may be saturated when the peripheral part of the image is dark if the brightness control process is performed over the entire field of view. According to the first embodiment, since weight is attached to the center part of the image when the observation magnification is high, the brightness of the center part of the field of view can be adjusted to an appropriate value. In particular, since the optical system illustrated in FIG. 3 magnifies only the center part of the field of view, it is likely that the doctor pays attention to the center part of the field of view.

The brightness control section 210 may include an evaluation value acquisition section (sampling section 402 illustrated in FIG. 5). The evaluation value acquisition section may set an acquisition target pixel or an acquisition target area (sampling area SA) for acquiring an evaluation value (average value Avg) of the brightness to the image, and may acquire the evaluation value based on the pixel value of the acquisition target pixel or the acquisition target area that has been set to the image (see FIG. 6, for example). The evaluation value acquisition section may perform a process that sets the acquisition target pixel or the acquisition target area to at least the center part of the image, and acquires the evaluation value as the photometric process when the observation magnification is the second magnification (see FIGS. 7 and 8, and the first to third modifications). The brightness control section 210 may adjust the brightness of the image based on the evaluation value.

It is possible to implement the photometric process that attaches weight to the center part of the image by thus setting the acquisition target pixel or the acquisition target area to at least the center part of the image, and acquiring the evaluation value. Specifically, the evaluation value that attaches weight to the center part of the image can be acquired by controlling the acquisition target pixel/acquisition target area setting method corresponding to the observation magnification.

More specifically, the evaluation value acquisition section (sampling section 402) may set a larger number of acquisition target pixels or acquisition target areas (sampling areas SA) to the center part of the image as compared with the part of the image other than the center part when the observation magnification is the second magnification.

The brightness control section 210 may include an area setting section (sampling section 402 illustrated in FIG. 5), for example. The area setting section may set the center area R1 and the peripheral area R2 to the image, the center area R1 corresponding to the center part of the image, and the peripheral area R2 being an area positioned outside the center area R1 (see FIG. 7). The evaluation value acquisition section may set the acquisition target pixel or the acquisition target area to the center area R1 and the peripheral area R2 when the observation magnification is the first magnification that is lower than a given magnification (magnification threshold value), and may set the acquisition target pixel or the acquisition target area to the center area R1 without setting the acquisition target pixel or the acquisition target area to the peripheral area R2 when the observation magnification is the second magnification that is higher than the given magnification.

It is possible to implement the brightness control process that attaches weight to the center part of the image by thus controlling the number of acquisition target pixels or acquisition target areas from which the pixel value is sampled. For example, the brightness control process can be switched between the brightness control process that takes account of the entire image and the brightness control process that attaches weight to the center part of the image by setting the number of acquisition target pixels or acquisition target areas set to the peripheral area R2 to a positive number or zero.

The evaluation value acquisition section may increase the density of (decrease the interval between) the acquisition target pixels or the acquisition target areas in the center part of the image as compared with the part of the image other than the center part when the observation magnification is the second magnification.

Note that the density of the acquisition target pixels or the acquisition target areas refers to the ratio of the number of acquisition target pixels or acquisition target areas set to a given area to the area of the given area. The density of the acquisition target pixels or the acquisition target areas may be the ratio of the number of pixels included in the acquisition target pixels or the acquisition target areas set to a given area to the total number of pixels included in the given area.

The evaluation value acquisition section may set the acquisition target pixels or the acquisition target areas to the center area R1 and the peripheral area R2 at an identical density (interval D) when the observation magnification is the first magnification that is lower than a given magnification, and may set the acquisition target pixels or the acquisition target areas to the center area R1 at a density higher than that of the peripheral area R2 (interval 2D) when the observation magnification is the second magnification that is higher than the given magnification (see the first modification (FIG. 8)).

It is possible to implement the brightness control process that attaches weight to the center part of the image by thus controlling the density of the acquisition target pixels or the acquisition target areas from which the pixel value is sampled. For example, the brightness control process can be switched between the brightness control process that takes account of the entire image and the brightness control process that attaches weight to the center part of the image by changing the ratio of the density of the acquisition target pixels or the acquisition target areas set to the center area R1 to the density of the acquisition target pixels or the acquisition target areas set to the peripheral area R2 (e.g., 1:1 or 2:1).

The evaluation value acquisition section may increase the weighting coefficient that weights the pixel value of the acquisition target pixel or the acquisition target area in the center part of the image as compared with the part of the image other than the center part when the observation magnification is the second magnification, and may acquire the evaluation value based on the pixel value that is weighted by the weighting coefficient.

For example, the evaluation value acquisition section may set an identical weighting coefficient to the center area R1 and the peripheral area R2 when the observation magnification is the first magnification that is lower than a given magnification, and may set a larger weighting coefficient to the center area R1 as compared with the peripheral area R2 when the observation magnification is the second magnification that is higher than the given magnification (see the second modification (expression (1))).

The evaluation value acquisition section may set the weighting coefficient that increases as the distance from the center point of the image decreases when the observation magnification is the second magnification (see the third modification).

It is possible to implement the brightness control process that attaches weight to the center part of the image by thus controlling the weighting coefficient by which the sampled pixel value is multiplied. For example, the brightness control process can be switched between the brightness control process that takes account of the entire image and the brightness control process that attaches weight to the center part of the image by changing the ratio of the weighting coefficient set to the center area R1 to the weighting coefficient set to the peripheral area R2 (e.g., Wc:Ws), or changing the weighting coefficient distribution with respect to the distance from the center point of the image.

The brightness control section 210 may adjust the brightness of the image by controlling at least one of the intensity of illumination light and a digital gain of the image based on the result of the photometric process.

For example, the light source control section 209 controls the intensity of illumination light by controlling the intensity of light emitted from the light source section 104 (see the expressions (2) and (3)), and the digital gain control section 205 controls the digital gain (see the expression (4)).

This makes it possible to perform the brightness control process based on the evaluation value (average value Avg) that is calculated while attaching weight to the center part of the image. When controlling both the intensity of illumination light and the digital gain, the brightness of the image can be further increased even if the intensity of illumination light is a maximum.

### 2. Second Embodiment

### 2.1. Endoscope Apparatus

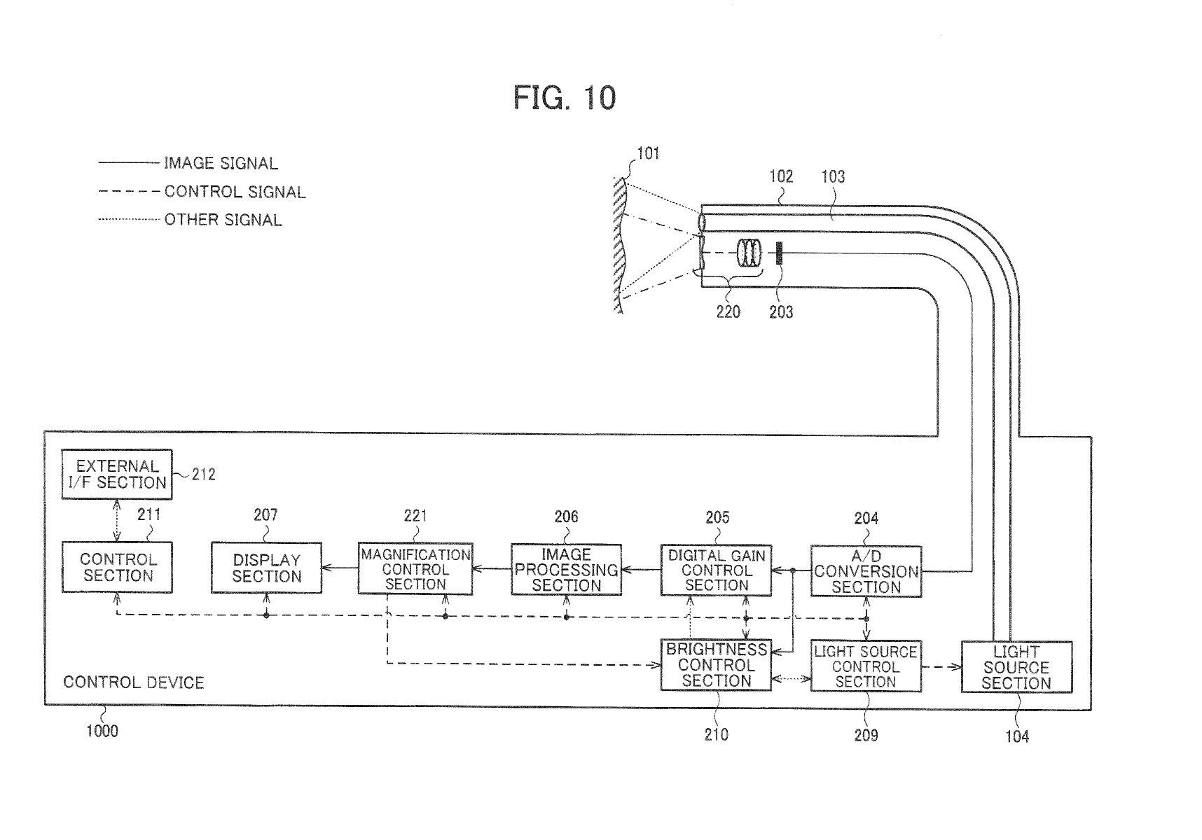

FIG. 10 illustrates a configuration example of an endoscope apparatus according to the second embodiment of the invention. As illustrated in FIG. 10, the endoscope apparatus includes an insertion section 102 and a control device 1000. The insertion section 102 includes a light guide 103, an image sensor 203, and an objective optical system 220 (objective lens). The control device 1000 includes a light source section 104, an A/D conversion section 204, a digital gain control section 205, an image processing section 206, a display section 207, a light source control section 209, a brightness control section 210, a control section 211, an external I/F section 212, and a magnification control section 221.

Note that the same elements as those described above in connection with the first embodiment are respectively indicated by the same reference signs, and description thereof is appropriately omitted.

The digital gain control section 205 is connected to the display section 207 via the image processing section 206 and the magnification control section 221. The magnification determination section 221 is connected to the brightness control section 210. The control section 211 is bidirectionally connected to the A/D conversion section 204, the digital gain control section 205, the image processing section 206, the display section 207, the magnification control section 221, the light source control section 209, the brightness control section 210, and the external I/F section 212.

The objective optical system 220 is a fish-eye lens that has a spatial imaging range of more than 180°. In the first embodiment, the center area and the peripheral area of the image respectively correspond to the field of view of the first optical system and the field of view of the second optical system. In the second embodiment, the center area and the peripheral area of the image are areas obtained by dividing the image into a center part and a peripheral part along a given boundary.

The magnification control section 221 controls the observation magnification by performing an electronic zoom process on the image. FIG. 12 illustrates a detailed configuration example of the magnification control section 221. The magnification control section 221 includes a magnification determination section 302, a ROM 303, and an electronic zoom magnification control section 601.

The image processing section 206 is connected to the brightness control section 210 via the electronic zoom magnification control section 601 and the magnification determination section 302. The ROM 303 is connected to the magnification determination section 302. The electronic zoom magnification control section 601 is connected to the display section 207. The control section 211 is bidirectionally connected to the electronic zoom magnification control section 601, the magnification determination section 302, and the ROM 303.

The electronic zoom magnification control section 601 performs image processing that zooms the image input from the image processing section 206, and transmits the resulting image to the display section 207. The zoom magnification corresponds to the observation magnification. The user instructs the observation magnification via the external I/F section 212. The control section 211 controls the electronic zoom magnification control section 601 based on the instruction input by the user. The magnification determination section 302 compares the observation magnification acquired from the electronic zoom magnification control section 601 with a magnification threshold value acquired from the ROM 303 under control of the control section 211. The magnification determination section 302 transmits the comparison result to the brightness control section 210. Note that the magnification threshold value differs depending on the type of the endoscopic scope.

The brightness control section 210 performs the brightness control process that attaches weight to the center part of the image in the same manner as in the first embodiment when the observation magnification is larger than the magnification threshold value. Although the observation range has no boundary when using the fish-eye lens, the center area R1 that corresponds to the center part of the observation range and the peripheral area R2 that corresponds to the peripheral part of the observation range are set to the image. The area setting information may be stored in the ROM 405, and the sampling section 402 may read the area setting information, and perform the sampling process. Note that the brightness control process is not limited to the brightness control process that sets the center area R1. It is also possible to perform the brightness control process that increases the weighting coefficient as the distance from the center pixel of the image decreases (see the third modification of the first embodiment).

Although the first and second embodiments illustrate an example in which the objective optical system has an angle of view of more than 180°, the first and second embodiments may also be applied to an objective optical system that has an angle of view equal to or less than 180°.

According to the second embodiment, the brightness of the center part of the field of view can be adjusted to an appropriate value when using a fish-eye lens by performing the brightness control process that attaches weight to the center part of the image when the observation magnification is high. Therefore, since the brightness control process can be adaptively performed corresponding to the observation state of the doctor, it is possible to contribute to an improvement in visibility of a lesion area.

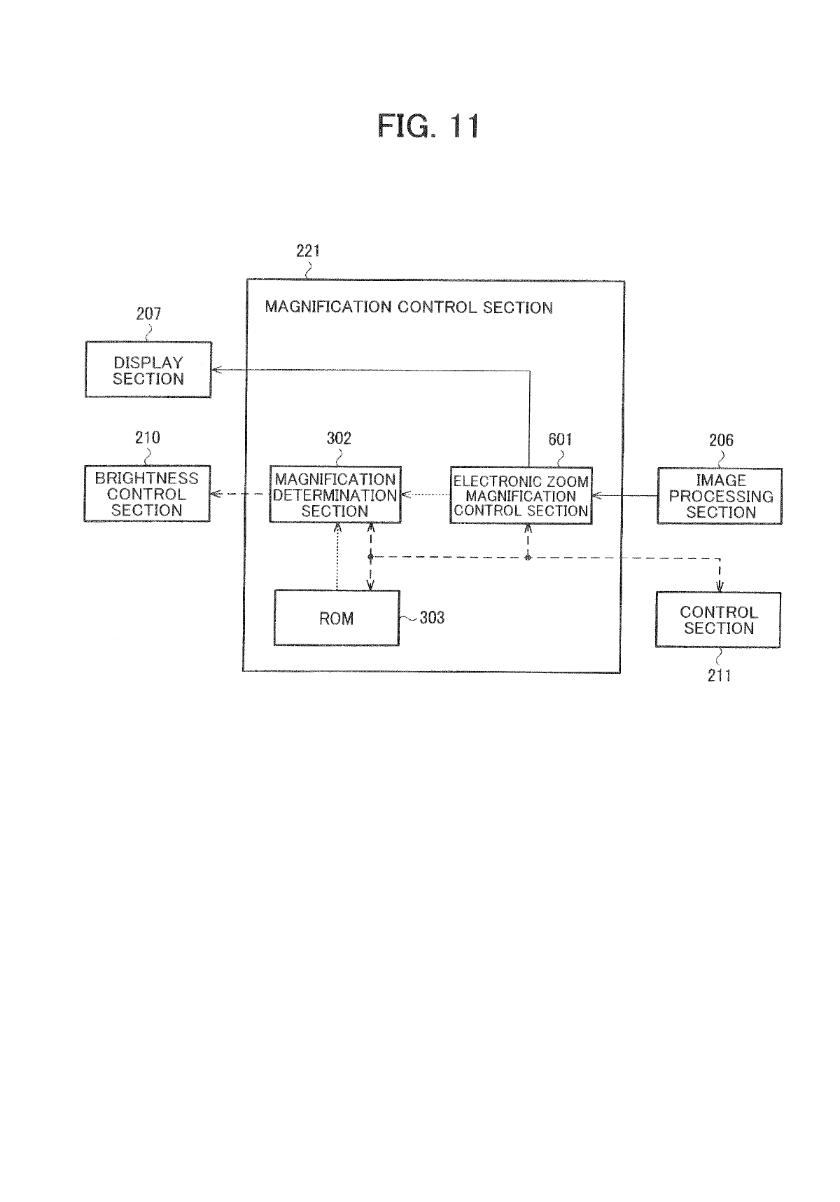

In the second embodiment, the magnification control section 208 may include at least one of the optical system magnification control section 301 (see FIG. 4) that controls the observation magnification of the objective optical system, and the electronic zoom magnification control section 601 (see FIG. 11) that controls the magnification of the electronic zoom process performed on the image.

This makes it possible to variably control the observation magnification using at least one of the optical zoom process and the electronic zoom process. Although the first and second embodiments illustrate an example in which the optical system illustrated in FIG. 3 and the optical zoom process, or the fish-eye lens and the electronic zoom process are used in combination, the optical system illustrated in FIG. 3 and the electronic zoom process, or the fish-eye lens and the optical zoom process may be used in combination. Alternatively, the optical zoom process and the electronic zoom process may be used in combination. In this case, the observation magnification is a value obtained by multiplying the optical zoom magnification by the electronic zoom magnification.

The embodiments to which the invention is applied and the modifications thereof have been described above. Note that the invention is not limited to the above embodiments and the modifications thereof. Various modifications and variations may be made without departing from the scope of the invention. A plurality of elements described in connection with the above embodiments and the modifications thereof may be appropriately combined to implement various configurations. For example, some of the elements described in connection with the above embodiments and the modifications thereof may be omitted. Some of the elements described in connection with different embodiments or modifications thereof may be appropriately combined. It is thus possible to implement various modifications and applications without materially departing from the novel teachings and advantages of the invention.

Any term cited with a different term having a broader meaning or the same meaning at least once in the specification and the drawings can be replaced by the different term in any place in the specification and the drawings.

What is claimed is:

1. An endoscope apparatus comprising:
an objective optical system that is configured so that an observation magnification in at least a center part of an observation range is variable;
a magnification control section that controls the observation magnification; and
a brightness control section that performs a photometric process that attaches weight to a center part of an image that corresponds to the center part of the observation range when the observation magnification is a second magnification that is higher than a first magnification as compared with a case where the observation magnification is the first magnification, and adjusts brightness of the image based on a result of the photometric process.

2. The endoscope apparatus as defined in claim 1,
the brightness control section including an evaluation value acquisition section that sets an acquisition target pixel or an acquisition target area for acquiring an evaluation value of the brightness to the image, and acquires the evaluation value based on a pixel value of the acquisition target pixel or the acquisition target area that has been set to the image,
the evaluation value acquisition section performing a process that sets the acquisition target pixel or the acquisition target area to at least the center part of the image, and acquires the evaluation value as the photometric process when the observation magnification is the second magnification, and
the brightness control section adjusting the brightness of the image based on the evaluation value.

3. The endoscope apparatus as defined in claim 2,
the evaluation value acquisition section setting a larger number of the acquisition target pixels or the acquisition target areas to the center part of the image as compared with a part of the image other than the center part when the observation magnification is the second magnification.

4. The endoscope apparatus as defined in claim 3,
the brightness control section including an area setting section that sets a center area and a peripheral area to the image, the center area corresponding to the center part of the image, and the peripheral area being an area positioned outside the center area, and
the evaluation value acquisition section setting the acquisition target pixel or the acquisition target area to the center area and the peripheral area when the observation magnification is the first magnification that is lower than a given magnification, and setting the acquisition target pixel or the acquisition target area to the center area without setting the acquisition target pixel or the acquisition target area to the peripheral area when the observation magnification is the second magnification that is higher than the given magnification.

5. The endoscope apparatus as defined in claim 2,
the evaluation value acquisition section increasing a density of the acquisition target pixels or the acquisition target areas in the center part of the image as compared with a part of the image other than the center part when the observation magnification is the second magnification.

6. The endoscope apparatus as defined in claim 5,
the brightness control section including an area setting section that sets a center area and a peripheral area to the image, the center area corresponding to the center part of the image, and the peripheral area being an area positioned outside the center area, and
the evaluation value acquisition section setting the acquisition target pixels or the acquisition target areas to the center area and the peripheral area at an identical density when the observation magnification is the first magnification that is lower than a given magnification, and setting the acquisition target pixels or the acquisition target areas to the center area at a density higher than that of the peripheral area when the observation magnification is the second magnification that is higher than the given magnification.

7. The endoscope apparatus as defined in claim 2,
the evaluation value acquisition section increasing a weighting coefficient that weights the pixel value of the acquisition target pixel or the acquisition target area in the center part of the image as compared with a part of the image other than the center part when the observation magnification is the second magnification, and acquiring the evaluation value based on the pixel value that is weighted by the weighting coefficient.

8. The endoscope apparatus as defined in claim 7,
the brightness control section including an area setting section that sets a center area and a peripheral area to the image, the center area corresponding to the center part of the image, and the peripheral area being an area positioned outside the center area, and
the evaluation value acquisition section setting an identical weighting coefficient to the center area and the peripheral area when the observation magnification is the first magnification that is lower than a given magnification, and setting a larger weighting coefficient to the center area as compared with the peripheral area when the observation magnification is the second magnification that is higher than the given magnification.

9. The endoscope apparatus as defined in claim 7,
the evaluation value acquisition section setting the weighting coefficient that increases as a distance from a center point of the image decreases when the observation magnification is the second magnification.

10. The endoscope apparatus as defined in claim 1,
the brightness control section adjusting the brightness of the image by controlling at least one of an intensity of illumination light and a digital gain of the image based on the result of the photometric process.

11. The endoscope apparatus as defined in claim 1,
the objective optical system having an angle of view of more than 180°.

12. The endoscope apparatus as defined in claim 11,
the objective optical system being a fish-eye lens.

13. The endoscope apparatus as defined in claim 12,
the brightness control section including:
a storage section that stores area setting information for setting a center area and a peripheral area, the center area corresponding to the center part of the image, and the peripheral area being an area positioned outside the center area; and
an area setting section that sets the center area and the peripheral area to the image based on the area setting information.

14. The endoscope apparatus as defined in claim 11,
the objective optical system including:
a lens optical system that forms an image of the center part of the observation range; and
a catoptric system that forms an image of a part of the observation range that is positioned outside the center part.

15. The endoscope apparatus as defined in claim 14,
the brightness control section including:
a storage section that stores area setting information for setting a center area and a peripheral area, the center area corresponding to the observation range for which an image is formed by the lens optical system, and the peripheral area corresponding to the observation range for which an image is formed by the catoptric system; and
an area setting section that sets the center area and the peripheral area to the image based on the area setting information.

16. The endoscope apparatus as defined in claim 1,
the magnification control section including a magnification determination section that determines whether the observation magnification is the first magnification that is lower than a given magnification, or the second magnification that is higher than the given magnification, and
the brightness control section performing the photometric process based on a determination result of the magnification determination section.

17. The endoscope apparatus as defined in claim 1,
the magnification control section including at least one of an optical system magnification control section that controls the observation magnification of the objective optical system, and an electronic zoom magnification control section that controls a magnification of an electronic zoom process performed on the image.

18. A brightness control method comprising:
controlling an observation magnification of an objective optical system that is configured so that the observation magnification in at least a center part of an observation range is variable; and
performing a photometric process that attaches weight to a center part of an image that corresponds to the center part of the observation range when the observation magnification is a second magnification that is higher than a first magnification as compared with a case where the observation magnification is the first magnification, and adjusting brightness of the image based on a result of the photometric process.
